(19) United States
(12) Patent Application Publication
(10) Pub. No.: US 2023/0238151 A1
(43) Pub. Date: Jul. 27, 2023

(54) DETERMINING A MEDICAL PROFESSIONAL HAVING EXPERIENCE RELEVANT TO A MEDICAL PROCEDURE

(71) Applicant: KONINKLIJKE PHILIPS N.V., EINDHOVEN (NL)

(72) Inventors: Grzegorz Andrzej Toporek, Cambridge, MA (US); Raghavendra Srinivasa Naidu, Auburndale, MA (US)

(21) Appl. No.: 17/918,928

(22) PCT Filed: Apr. 15, 2021
(86) PCT No.: PCT/EP2021/059717

§ 371 Date: Oct. 14, 2022

Related U.S. Application Data
(60) Provisional application No. 63/010,787, filed on Apr. 16, 2020.

(51) Int. Cl. G16H 80/00; G16H 20/40

(52) U.S. Cl. CPC G16H 80/00; G16H 20/40

(57) ABSTRACT

Systems and methods of determining a second medical professional having experience relevant to a medical procedure being performed by a first medical professional. A method of determining a second medical professional having experience relevant to a medical procedure being performed by a first medical professional comprises: obtaining a first multi-dimensional embedding vector related to the medical procedure being performed by the first medical professional. The method then comprises obtaining a plurality of candidate multi-dimensional embedding vectors related to medical experience of a corresponding plurality of candidate medical professionals, and using a model to determine the second medical professional from the plurality of candidate medical professionals, based on the first multi-dimensional embedding vector and the plurality of candidate multi-dimensional embedding vectors.

### TECHNICAL FIELD

The disclosure herein relates to medical teleconference systems. Particularly, but non-exclusively, embodiments herein relate to systems and methods for determining a second medical professional having experience relevant to a medical procedure being performed by a first medical professional.

### BACKGROUND

Portable medical devices, such as portable ultrasound scanners are of increasing interest in point-of-care settings. Portable medical devices may be used, for example, by medical professionals in emergency departments (ED), intensive care units (ICU), in bed-side examinations, or in other scenarios where a patient is examined in situ. The use of portable scanners, such as portable ultrasound scanners can add increased confidence to a medical professional's diagnostic decision making, and thus improve clinical outcomes.

### SUMMARY

As described above, the use of portable medical devices such as portable scanners can improve patient outcomes, especially when a patient is diagnosed and treated in the field (e.g. in situ; away from a medical facility setting). However, the medical professionals expected to perform imaging using said portable scanners may not be fully trained in performing a scan or interpreting their results. Lack of an onsite expert may result in discomfort using such portable equipment and this may result in low uptake or usage. In remote locations, such as in the countryside, or in countries where the population may be dispersed and patients may be unable to travel to a medical facility (such as in Africa), medical professionals may need feedback or input from another medical professional in order to perform or interpret the findings from a remote examination.

Medical teleconferencing (e.g. real-time audio or video calls) may be used to connect remote medical professionals (e.g. the users of portable medical devices) with their colleagues at other locations, with the aim of assisting remote diagnostic decision making, or even to help the user acquire appropriate data sets.

Current medical teleconferencing systems connect medical professionals with other experts from a previously defined contact list. They may further rely on online availability of these users. This leads to the challenge of quickly looking for available experts in the contact list. Moreover in emergency settings, the user may be flustered and require quicker decision making and quicker accessibility to an expert clinician. It is thus an object of embodiments herein to provide improved support to remote medical professionals.

In particular, but non-exclusively, systems and methods herein enable additional information such as case-specific experience, work-load, and outcomes of previous remote sessions to be taken into account when indicating possible experts with whom a remote medical professional may want to set up a call. Some embodiments herein thus relate to real-time recommendation systems for remote tele-collaboration tools integrated into medical imaging modalities. As will be described in more detail, in some embodiments herein, information extracted during imaging, such as image quality, diagnostic outcomes, characteristics of user-device interactions, as well as ratings from previous remote sessions, are used to propose an ordered set of expert users (e.g. potential collaborators), whose advice may be the most beneficial to the (remote) medical professional performing the medical procedure.

Thus, according to a first aspect, there is a system for determining a second medical professional having experience relevant to a medical procedure being performed by a first medical professional. The system comprises a memory comprising instruction data representing a set of instructions; and a processor configured to communicate with the memory and to execute the set of instructions, wherein the set of instructions, when executed by the processor, cause the processor to: obtain a first multi-dimensional embedding vector related to the medical procedure being performed by the first medical professional; obtain a plurality of candidate multi-dimensional embedding vectors related to medical experience of a corresponding plurality of candidate medical professionals; and use a model to determine the second medical professional from the plurality of candidate medical professionals, based on the first multi-dimensional embedding vector and the plurality of candidate multi-dimensional embedding vectors.

In this way the first medical professional may be matched to a second professional based on relevant medical experience to the medical procedure being performed by the first medical professional. This results in improved recommendations, improved assistance provided to the first medical professional and improved patient outcomes.

According to a second aspect there is a computer implemented method of determining a second medical professional having experience relevant to a medical procedure being performed by a first medical professional. The method comprises obtaining a first multi-dimensional embedding vector related to the medical procedure being performed by the first medical professional; obtaining a plurality of candidate multi-dimensional embedding vectors related to medical experience of a corresponding plurality of candidate medical professionals; and using a model to determine the second medical professional from the plurality of candidate medical professionals, based on the first multi-dimensional embedding vector and the plurality of candidate multi-dimensional embedding vectors.

According to a third aspect, there is a computer program product comprising computer readable medium, the computer readable medium having computer readable code embodied therein, the computer readable code being configured such that, on execution by a suitable computer or processor, the computer or processor is caused to perform the method as in the second aspect.

### BRIEF DESCRIPTION OF THE DRAWINGS

For a better understanding and to show more clearly how embodiments herein may be carried into effect, reference will now be made, by way of example only, to the accompanying drawings, in which:

FIG. 1 shows an example system according to some embodiments herein;

FIG. 2a illustrates a method of training of a neural network according to some embodiments herein;

FIG. 2b shows inference of a neural network according to some embodiments herein;

FIG. 3 shows an example matrix according to some embodiments herein;

FIG. 4 shows an example system according to some embodiments herein;

FIG. 5 shows an example graphical user interface according to some embodiments herein;

FIG. 6 shows further example graphical user faces according to some embodiments herein; and

FIG. 7 shows an example method according to some embodiments herein;

### DETAILED DESCRIPTION

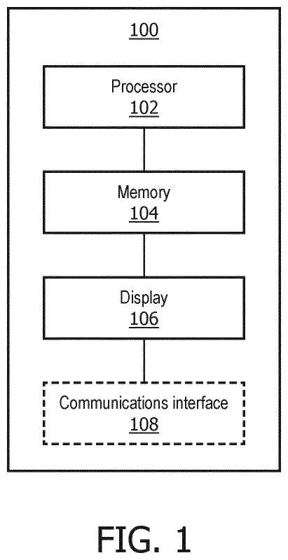

FIG. 1 illustrates a system (e.g. apparatus) 100 for determining a second medical professional having experience relevant to a medical procedure being performed by a first medical professional, according to some embodiments herein.

Generally, the system 100 may comprise a stand-alone system, or be comprised in (or form part of) another system or apparatus. The system 100 may be comprised in a telecommunications system, such as a medical telecommunications system. Generally, therefore, the system 100 may form part of equipment for performing audio or video calls.

In some embodiments, the system 100 may be comprised in a medical device. For example, the system 100 may be comprised in a telecommunications system that is integrated into a medical device. As such the system 100 may be used to recommend a second medical professional to the first medical professional as someone who may be able to provide assistance to the first medical professional when performing a medical procedure with the medical device. In some embodiments, the system 100 may be comprised in medical scanning or imaging equipment, such as an ultrasound scanner. Such an ultrasound scanner may be a 2D or 3D ultrasound scanner. Examples of other medical equipment that the system 100 may be comprised in include but are not limited to a monocular or stereo camera for skin cancer detection; equipment for performing diabetic retinopathy detection from retina images; and a sensing system such as a hyperspectral camera, or blood sampling system, etc.

The system 100 may further be comprised in an interventional ultrasound catheter (intravascular, intracardiac ultrasound). Other types of medical scanning (e.g. medical imaging) equipment include but are not limited to CT, MRI, or PET-CT and X-ray systems. In some embodiments, the medical device may comprise a portable medical device, e.g. a medical device designed to be used outside the hospital setting, e.g. in the patient's home, or at the location where a patient needs medical attention. Examples of portable medical devices include, but are not limited to, portable scanners, such as a portable ultrasound (or any other type of) scanner.

In other embodiments, the system 100 may comprise part of a review-based system. For example, the system 100 may be used to select a second medical professional having experience relevant to the medical procedure being performed by the first medical professional, in order that the second medical professional can review the performance of the first medical professional, e.g. peri-interventional review by a colleague physically present at the hospital facility (who was notified by the system), or post-procedural review by an expert at the end of the day.

With reference to FIG. 1, the system 100 comprises a processor 102 that controls the operation of the system 100 and that can implement the method described herein. The processor 102 can comprise one or more processors, processing units, multi-core processors or modules that are configured or programmed to control the system 100 in the manner described herein. In particular implementations, the processor 102 can comprise a plurality of software and/or hardware modules that are each configured to perform, or are for performing, individual or multiple steps of the method described herein.

In some embodiments, as illustrated in FIG. 1, the system 100 may also comprise a memory 104 configured to store program code that can be executed by the processor 102 to perform the method described herein. Alternatively or in addition, one or more memories 104 may be external to (i.e. separate to or remote from) the system 100. For example, one or more memories 104 may be part of another device. A memory 104 can be used to store images, information, data, signals and measurements acquired or made by the processor 102 of the system 100 or from any interfaces, memories or devices that are external to the system 100.

In some embodiments, as illustrated in FIG. 1, the system 100 may also comprise at least one user interface such as a user display 106. The processor 102 may be configured to control the user display 106 to display or render, for example, details of the determined second medical professional, information related to the medical procedure being performed by the first medical professional, and/or any of the outputs of the methods described herein. The user display 106 may comprise a touch screen or an application (for example, on a tablet or smartphone), a display screen, a graphical user interface (GUI) or other visual rendering component.

Alternatively or in addition, at least one user display 106 may be external to (i.e. separate to or remote from) the system 100. For example, at least one user display 106 may be part of another device. In such embodiments, the processor 102 may be configured to send an instruction (e.g. via a wireless or wired connection) to the user display 106 that is external to the system 100 in order to trigger (e.g. cause or initiate) the external user display(s) to display information related to or resulting from the method described herein.

In some embodiments, the system 100 may comprise a Communications Interface 108 for enabling the system 100 to communicate with any interfaces, memories and devices that are internal or external to the system 100, for example over a wired or wireless network. Communications interface 108 may be used to provide a wired or wireless connection to send information or data from the system 100, to other devices, or systems (e.g. such as the cloud). The communications interface may be used to set up an audio-visual connection (e.g. a call) with a device operated by the second medical professional according to the methods herein.

As noted above, in some embodiments herein, the system 100 may be comprised in a medical device, or medical equipment and thus the system 100 may comprise further elements related to the operation of said medical device. For example, in some embodiments, the system 100 may be comprised in a portable ultrasound equipment. As such, the system 100 may further comprise components configured to capture and/or process ultrasound images. For example, in some embodiments, the system 100 may further comprise a transducer for capturing ultrasound images. Such a transducer may be formed from a plurality of transducer elements. Such transducer elements may be arranged to form an array of transducer elements. A transducer may be comprised in a probe such as a handheld probe that can be held by a user (e.g. sonographer, radiologist or other clinician) and moved over a patient's skin. The skilled person will be familiar with the principles of ultrasound imaging, but in brief, ultrasound transducers comprise piezoelectric crystals that can be used both to generate and detect/receive sound waves. Ultrasound waves produced by the ultrasound transducer pass into the patient's body and reflect off the underlying tissue structures. Reflected waves (e.g. echoes) are detected by the transducer and compiled (processed) by a computer to produce an ultrasound image of the underlying anatomical structures, otherwise known as a sonogram. In some embodiments a transducer may comprise a matrix transducer that may interrogate a volume space.

It will be appreciated that FIG. 1 only shows the components required to illustrate this aspect of the disclosure, and in a practical implementation the system 100 may comprise additional components to those shown. For example, the system 100 may comprise other user interfaces for receiving inputs from a user, such as a mouse, button or touch-screen device. The system 100 may further comprise a battery or other means for connecting the system 100 to a mains power supply.

Briefly, the processor 102 of the system 100 is configured to obtain a first multi-dimensional embedding vector related to the medical procedure being performed by the first medical professional; obtain a plurality of candidate multi-dimensional embedding vectors related to medical experience of a corresponding plurality of candidate medical professionals; and use a model to determine the second medical professional from the plurality of candidate medical professionals, based on the first multi-dimensional embedding vector and the plurality of candidate multi-dimensional embedding vectors.

Technically, this may provide an improved manner in which to recommend a second medical professional to the first medical professional, based on the experience and relevance of the experience of the second medial professional for a specific medical procedure.

Herein the first medical professional may be any type of clinician, doctor, nurse, sonographer, radiologist, paramedic, general user of medical equipment, or any other person performing a medical procedure.

Generally the medical procedure may comprise any type of procedure, examination, assessment, or any other consultation (e.g. where the first medical professional requires assistance from a second medical professional). In some embodiments, herein, for example, the medical procedure may comprise an ultrasound examination, or an examination requiring medical imaging of any of the imaging modes described above. The first medical professional may be performing the medical procedure at a remote location, e.g. away from a medical facility. As noted above the medical procedure may comprise an emergency procedure where urgent assistance from a second medical professional is sought.

As noted above, the processor 102 is caused to (e.g. configured to) obtain a first multi-dimensional embedding vector related to the medical procedure being performed by the first medical professional. The skilled person will be familiar with multi-dimensional embedding vectors which comprise a manner in which to map (e.g. represent or convert) information (such as data, objects or images) to vectors of real numbers.

Multi-dimensional embedding vectors may be manually or automatically extracted from images (e.g. such as images acquired during an ultrasound scanning session), using methods that will be familiar to the skilled person. For instance, live ultrasound images may be classified in terms of image quality, anatomical content, and diagnostic outcomes using approaches including, for example, segmentation, SNR, and manual user input. In some embodiments live ultrasound images may be classified using machine learning approaches such as deep convolutional neural networks, or any machine learning methods including support vector machine, decision tree, multilayer perceptron, or multivariate regression classifiers. As another example, the type of procedure being performed may be identified by applying object detection algorithms to images obtained from a camera (e.g. such as a tablet computer's back-facing camera), as well as text analysis of the generated reports, voice commands from the operator, and characteristics of the operator. Object detection algorithms may be used, for example, to make a classification related to the patient's state, e.g. whether the patient is obese vs. non-obese, patient position (e.g. supine, lateral), and/or probe position in respect to the patient. Such classifications/information may be enumerated and combined to produce a multi-dimensional embedding vector.

Generally, the first multi-dimensional embedding vector may comprise a multi-dimensional vector related to the medical procedure being performed by the first medical professional. It may comprise (e.g. be calculated, or based on) any aspect related to the medical procedure being performed by the first medical professional. For example, it may reflect the medical experience of the first medical professional, the type of medical procedure being performed, the environment or circumstances in which the medical procedure is being performed (e.g. remote or local, sanitation of the conditions), and/or the patient that the medical procedure is being performed on (e.g. patient's condition, responsiveness, physical characteristics, weight, position, etc).

In some embodiments, the first multi-dimensional embedding vector may comprise a signature of an image of the medical procedure being performed by the first medical professional. In other words, the first multi-dimensional embedding vector may be based on (or determined based on) images of the first medical professional performing the first medical procedure, images of the location, images of the patient or any other images of the environment at which the first medical professional is to perform or is performing the medical procedure. In some embodiments, the first medical professional may make an audio-visual recording of the location at which the medical procedure is being performed and the first multi-dimensional embedding vector may be based on one or more frames of the audio-visual recording.

In some embodiments, the signature may be obtained from an (arbitrary) feature map of a feed-forward convolutional neural network, CNN. For example, the first multi-dimensional embedding vector may comprise an image feature vector, extracted from a flattened feature map generated by a feed-forward pass of an input image over a convolutional neural network (CNN). Such a CNN may comprise one or a plurality of convolutional layers and at least one fully connected layer located after the intermediate or last convolutional layers. A convolutional layer may comprise, for example, a convolution operation, spatial pooling, dropout, batch normalization, and/or non-linearity. Values from the activated neurons in the fully connected layers define a signature of the image.

Alternatively, a dimensionality of a multidimensional feature map generated by the last intermediate convolutional layers may be reduced or reshaped to create a signature of the image (feature vector). Possible operations for dimensionality change or reduction may include global max or average pooling, or simple reshaping (flattening operation).

A CNN for such a purpose may be trained on an arbitrary task, such as classification, regression, data generation (autoencoders), or segmentation using large data set consisting of images acquired with specific imaging modality, such as ultrasound. In result, weights of this network are optimized to extract strong salient features. The output of any given layer can thus be compared between different images, forming a vector space with similar images being located in closer proximity in the vector space to dissimilar images.

In some embodiments, the first multi-dimensional embedding vector comprises information relating to a measure of image quality of an ultrasound cine loop, e.g. of ultrasound images obtained as part of the ultrasound examination.

In some embodiments, the first multi-dimensional embedding vector comprises information relating to the contents of an ultrasound cine loop of the medical procedure. For example, the first multi-dimensional embedding vector may be based on an anatomical feature visible in an ultrasound cine loop of the procedure being performed by the first medical professional. For example, a descriptor of the organ or area of interest, such as abdominal (liver, kidney) or thoracic (lung) cavity. In some embodiments, the first multi-dimensional embedding vector comprises information relating to diagnostic evaluation of the medical procedure as deduced from an ultrasound cine loop of the procedure being performed by the first medical professional. For example, such as presence of malignant vs. benign lesion. Classification of the image in this manner may be performed in an automated manner, for example, using machine learning approaches, or manually by the user. Such classification may comprise a multi-label classification of the image, for example, combining any of the elements discussed above. In this way medical context information may be taken into account when matching the second medical professional to the first medical professional.

In some embodiments, the first multi-dimensional embedding vector comprises information relating to a measure of image quality of a video stream of the procedure being performed by the first medical professional. Such a video stream may be obtained from an optical camera.

In some embodiments, the first multi-dimensional embedding vector comprises information relating to the environment in which the medical procedure is being performed. For example, a scene descriptor may be generated from the medical environment, for example, using machine learning approaches such as multi-label classifier or object detection algorithms.

Scene descriptors may be extracted from a video stream (e.g. sequence of images) from the forward or back-facing cameras on mobile devices or tablets and may include, for example:

i. Objects present during the examination such as peripheral medical devices and modalities, additional medical staff,

ii. Patient characteristics, such as body weight, bleeding, bone fracture,

iii. Environment classification, such as indoor, outdoor examination.

Other examples include but are not limited to location characteristic (high-way, hospital, football field, day, night), which could influence the criticality of the expert intervention. A video stream may also detect patient characteristics, such as whether the patient is bleeding or experiencing pain from the facial expression, etc.

In this way, the first multi-dimensional embedding vector may comprise information about the environment which may enable the system to determine a second medical professional having experience of performing/advising on performing the medical procedure in similar environments (e.g. similar terrain or circumstances).

In some embodiments, the first multi-dimensional embedding vector comprises information relating to the experience or actions of the first medical professional. For example, user-device interaction characteristics. For example, in embodiments where the medical procedure comprises an ultrasound examination, the if the first multi-dimensional embedding vector may comprise information relating to user interaction with the medical imaging equipment used to perform the medical procedure, such as for example, a measured number of clicks by the user, user initiated changes in the imaging parameters, the motion of the transducer, from which one could derive experience of the user (novice vs. experienced; anxiety level). It is noted that motion of a transducer may be recorded using, for example, an Inertial measurement unit (IMU) sensor embedded in the transducer; and interactions with a graphical user interface (GUI) may be determined, for example, from log files. In this way the first multi-dimensional embedding vector may comprise information about the experience of the first medical professional which may enable the system to determine a second medical professional having complementary experience of advising medical professionals with similar levels of medical experience as the first medical professional.

The skilled person will appreciate however, that these are examples, and that the first multi-dimensional embedding vector may be determined or calculated based on any other factors, such as, for example, word embedding from manually or automatically generated reports, connection statistics describing a quality or connection type of an electronic communication or video-feed.

Essentially therefore, the first multi-dimensional embedding vector comprises a vector of numbers that acts as a “bar-code” or unique identifier for the medical procedure being performed by the first medical professional. It will be appreciated that the first multi-dimensional embedding vector may comprise any combination of options described above. Furthermore, the first multi-dimensional embedding vector may comprise a plurality of multi-dimensional embedding vectors that are combined to form the first multi-dimensional embedding vector.

In some embodiments, the first multi-dimensional embedding vector may be determined (e.g. calculated) in real time by the processor 102.

The first multi-dimensional embedding vector may be manually or automatically extracted from a scanning session, using methods know in the art. For example, live ultrasound images may be classified in terms of image quality, anatomical content, and diagnostic outcomes using machine learning approaches such as deep convolutional neural networks. Furthermore, the medical procedure being performed may be classified (e.g. by type) by applying object detection algorithms to images, for example, images from a tablet computer's back-facing cameras. The medical procedure may also be classified or identified from text analysis of generated reports, and/or characteristics of the operator. The skilled person will appreciate however that these are merely examples and that the first multi-embedding vector may be determined in other manners to those listed herein.

The plurality of candidate medical professionals comprise a plurality of other medical professionals (e.g. other experts) who may be consulted in order to provide assistance to the first medical professional when performing the medical procedure. As such it may comprise a list of other medical professionals whom the first medical professional could contact for assistance. For each candidate medical professional, a candidate multi-dimensional embedding vector is determined (e.g. obtained or calculated). Each candidate multi-dimensional embedding vector relates to the medical experience of one of the candidate medical professionals in the plurality of candidate medical professionals. In some embodiments, it may be retrieved, e.g. from a database.

Each candidate multi-dimensional embedding vector may be based, for example, on: i) number of years of experience of the respective candidate medical professional, ii) primary area of expertise (e.g. abdominal, cardiac, gastric, focused assessment with sonography in trauma—FAST exam, etc.), iii) characteristics of previous medical procedures (or remote sessions) with which the candidate medical professional has assisted with.

The processor 102 is then configured to use a model to determine the second medical professional from the plurality of candidate medical professionals, based on the first multi-dimensional embedding vector and the plurality of candidate multi-dimensional embedding vectors. In other words, the processor 102 is be configured to match the first medical performing the medical procedure to a second medical professional, selected from a plurality of candidate medical professionals. In some embodiments, the second medical professional is predicted by the model as someone with experience relevant to the medical procedure, who may be able to assist the first medical professional.

As described briefly above, in some embodiments, the system comprises a medical teleconferencing system. The processor 102 may thus be configured to recommend the second medical professional to the first medical professional as a person with whom to initiate a video call in order to obtain remote assistance with the medical procedure.

In some embodiments, the model may comprise a model trained using a machine learning process. The model may be trained to take as input the first multi-dimensional embedding vector and a candidate multi-dimensional embedding vector from the plurality of candidate multi-dimensional embedding vectors, and output a relevance value for the respective candidate medical professional, wherein the relevance value comprises a prediction of the relevance of the respective candidate medical professional to the medical procedure being performed by the first medical professional.

The skilled person will be familiar with machine learning models and methods of training a machine learning model. In some embodiments, the model may comprise a supervised machine learning model. Examples of machine learning models that may be used herein include, but are not limited to decision tree (random forest) classifiers, neural networks models, multilayer perceptron classifiers, regression models, and support vector machine classifiers.

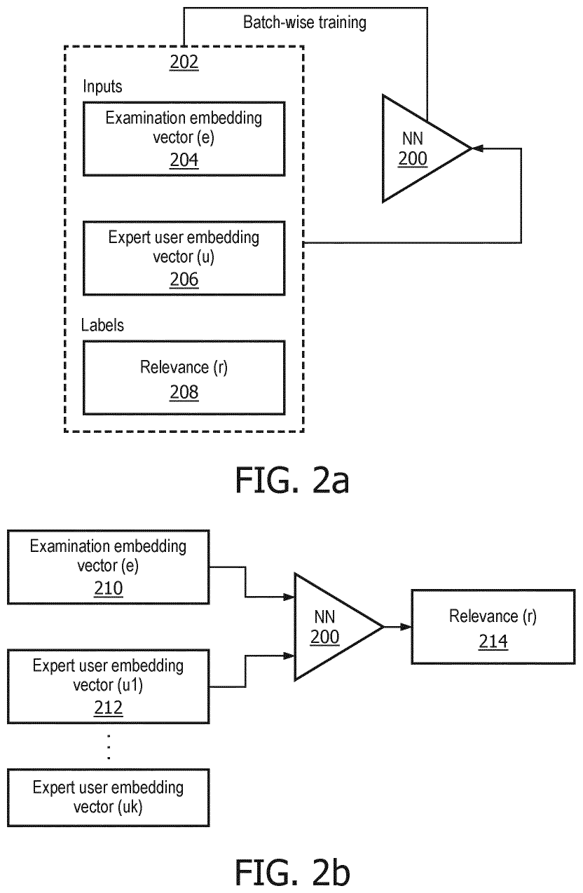

In some embodiments, the model may comprise a trained neural network model 200, such as a feed-forward neural network, as illustrated in FIG. 2. The skilled person will be familiar with neural networks, but in brief, neural networks are a type of machine learning model that can be trained to predict a desired output for given input data. Neural networks are typically trained by providing training data comprising example input data and the corresponding “correct” or ground truth outcome that is desired. Neural networks comprise a plurality of layers of neurons, each neuron representing a mathematical operation that is applied to the input data. The output of each layer in the neural network is fed into the next layer to produce an output. For each piece of training data, weights associated with the neurons are adjusted until the optimal weightings are found that produce predictions for the training examples that reflect the corresponding ground truths.

In embodiments wherein the trained model comprises a neural network, the neural network may comprise a convolutional neural network, consisting of one or plurality of intermediate convolutional layers as described above.

In some embodiments, training the model may comprise training the model on training data, comprising: i) a multi-dimensional embedding vector related to an example medical procedure ii) a multi-dimensional embedding vector related to medical experience of an example medical professional; and iii) a ground truth relevance value comprising a measure of relevance of the example medical professional to the example medical procedure.

The training data may comprise a large data set of multi-dimensional pairs of embedding vectors. The data set may be sampled from a large patient population, containing patients of different ages, genders, stages of disease, anatomical abnormalities, as well as different medical procedures, such as, for example, various diagnostic, pre- and peri-interventional imaging scans, such as transthoracic echocardiography of the heart, abdominal echo, transrectal ultrasound, or FAST exams. It may also comprise entries of a wide range of example medical professionals, such as experts with various area of expertise (cardiac, abdominal, intracranial), and experience (intermediate, well-trained), as well as backgrounds (emergency department, liver surgery, bed-side examinations).

The training data set may comprise many examples (e.g. hundreds, or thousands) of pieces of training data. As an example, a training data set may be in the following format: d=(e_{candidate}, e_{procedure}, r), where e_{candidate} stands for a multidimensional embedding vector of the candidate, e_{procedure} stands for a multidimensional embedding vector for the procedure, and r stands for the relevance rating (e.g. extracted from a user rating system). The embedding vectors may comprise a plurality of floating point numbers e.g. [0.16, −0.31, 0.23 . . . ]. For the image signature, the embedding vector could comprise a latent space in an autoencoder, and combination. Generally it can be 1 or n dimensional. For instance classification of the image quality could be embedded as the hot-one encoding: [0 1] High Quality and [1 0] for Low Quality.

In this example, the model is thus trained to predict the field: r from the input parameters: e_{candidate} and e_{procedure}T. he skilled person will appreciate however that this is merely an example, and that the training data may take other formats, or include other input or output fields in addition to or instead of those in this example.

In some embodiments, the ground truth relevance value comprises user feedback from a third medical professional with respect to the relevance of assistance provided by the example medical professional to the example medical procedure. For example, the medical professional who performed the medical procedure may initiate a call with a candidate medical professional in order to obtain assistance with an example medical procedure. After the call has terminated, the medical professional may rate or rank the candidate medical professional according to, for example, their usefulness or help provided during the call. This feedback may be used as a ground truth relevance value for the example medical procedure.

The skilled person will be familiar with methods of training a neural network using training data (e.g. gradient descent etc.) and appreciate that the training data may comprise many hundreds or thousands of rows of training data, obtained in a diverse range of network conditions.

FIG. 2a illustrates a schematic overview of an embodiment where the model comprises a neural network 200 that is trained using a batchwise training process. In this embodiment, the neural network may comprise, for example, a fully-connected multilayer perceptron or convolutional neural network. In this embodiment, the training process has many iterations. The training data comprises a plurality of tuples ((e, u), r) 202, each tuple 202 comprising i) a multi-dimensional embedding vector 204 related to an example medical procedure (referred to in this embodiment as “examination embedding vector e”) ii) a multi-dimensional embedding vector 206 related to medical experience of an example medical professional (referred to in this embodiment as “expert user embedding vector u”) and iii) a ground truth relevance value, r 208, comprising a measure of relevance of the example medical professional to the example medical procedure. At each iteration a batch consisting of multiple 3-tuples ((e, u), r) is randomly sampled from a large training data set and fed into a structure-predesigned neural network (e.g. a network with a fixed architecture). The network accepts examination embedding vector (e) and expert user embedding vector (u) 204, 206 at its input layer and transfers the ground truth relevance label (r) 208 to its output layer directly. The network is full of need-to-be-learned weights. At each iteration of the learning process, these weights are updated by propagating the errors between the predicted transformation and the ground truth one first in a backward direction and then in a forward direction. Training of the weights may be performed, for example, until the transformation predicted from the network is similar enough (e.g. within a threshold similarity) to the ground truth.

Inference is illustrated for this embodiment in FIG. 2b which illustrates a schematic overview of the real-time inference process using the trained neural network 200. A first multi-dimensional embedding vector 210 (e) (labelled as the “examination embedding vector” in FIG. 2b) related to the medical procedure being performed by the first medical professional is provided as input. The second input comprises a plurality of candidate multi-dimensional embedding vectors 212 (labelled “expert user embedding vectors” in FIG. 2b) related to medical experience of a corresponding plurality of candidate medical professionals. The candidate medical professional may comprise the set of k currently online expert users {u_l . . . u_k}. Trained neural network 200 predicts the relevance r\hat{ } 214 of each expert user in the list of candidate medical professionals—described by u—for the medical procedure being performed by the first medical professional. In this way, a neural network may be trained based on historical data and user feedback to recommend a second medical professional who may be able to assist a first medical professional with a medical procedure.

A neural network may be used in this way to predict relevance values for each of a plurality of candidate medical professionals to a medical procedure being performed by a first medical professional. In some embodiments, the processor may be configured to display a list comprising a subset of the plurality of candidate medical professionals to the first medical professional, the list being ordered according to relevance values of the subset of the plurality of candidate medical professionals as determined by the model. In this way, the model may be used to rank medical professionals and recommend medical professionals (in preferential order) with whom the medical professional may want to communicate with in order to seek assistance with the medical procedure.

Turning now to other embodiments, in some embodiments the processor 102 may be caused to perform collaborative filtering to determine a second medical professional with experience relevant to the medical procedure being performed by the first medical professional. For example, the processor being caused to use a model to determine the second medical professional from the plurality of candidate medical professionals may be further based on information relating to a plurality of previously performed medical procedures. The information may comprise, for each of the previously performed medical procedures: a multi-dimensional embedding vector, relating to a previous medical professional who performed the previous medical procedure; an indication of a third medical professional from the plurality of candidate medical professionals who assisted with the previous medical procedure; and a feedback rating provided by the previous medical professional that rates the assistance provided by the third medical professional to the previous medical procedure.

In some embodiments, the step of using a model to determine the second medical professional from the plurality of candidate medical professionals may thus comprise: using memory based collaborative filtering to determine the second medical professional based on the first multi-dimensional embedding vector and the information relating to the plurality of previously performed medical procedures.

The skilled person will be familiar with methods of collaborative filtering (CF) and variations of CF, such as, for example, CF neighbourhood or CF latent factors methods. CF methods provide recommendations e.g. recommend a second medical professional for the first medical professional to connect with, by finding previous medical procedures from the plurality of previously performed medical procedures that are most similar to the medical procedure being performed by the first medical professional. The CF model then recommends medical professionals from the plurality of medical professionals that were highly ranked by the previous medical professional who performed the most similar previous medical procedure.

In some embodiments, model-based collaborative filtering may be applied, using, for example, SVD, PCA, matrix factorization or neural network methods as described above with respect to FIG. 2. In other embodiments, memory-based collaborative filtering may be used, for example, using a similarity metric such as the Pearson correlation, Jaccard or cosine distance.

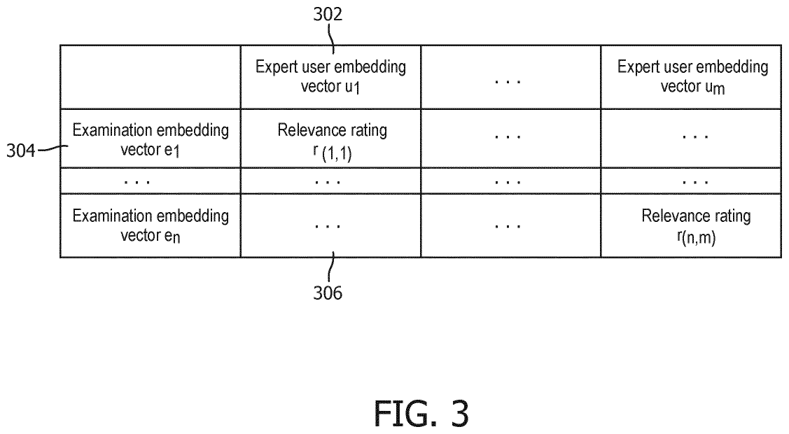

An embodiment is illustrated in FIG. 3 which illustrates an embodiment whereby the information relating to a plurality of previously performed medical procedures is arranged in a sparse matrix, with candidate multi-dimensional embedding vectors for the plurality of candidate medical professionals 302 (labelled “expert user embedding vector” uᵢ-uₘ. in FIG. 3) and multi-dimensional embedding vectors, relating to a previous medical professional, performing a previous medical procedure 304 (labelled “examination embedding vector” e₁-eₙ in FIG. 3) and feedback ratings 306 (labelled “relevance ratings” r_{(1,1)}-r_{(u,1)} in FIG. 3).

In this manner, the multi-dimensional embedding vectors may be combined into a utility matrix M∈R^{N×M}, where M is the number of candidate medical professionals (columns) and N is the number of previously performed medical procedures (rows) available in the database. As noted above, the entries of the matrix are filled in with relevance ratings or feedback that describes whether (or the degree to which) advice and support given by the candidate medical professionals users during the previous medical procedures were valuable.

The feedback ratings may be, for example, integers starting from to 1 to K ((r_{(u,i)}∈1 . . K). In some embodiments, it may be assumed that the feedback ratings are static. The feedback ratings may be provided by the previous medical professional who performed the previous medical procedure (e.g. the operator) of the imaging modality at the end of a remote teleconferencing session with the candidate medical professional, for example, using a EUR controller.

In other embodiments, clustering algorithms (such as, for example, k-means,

Gaussian mixture models-based clustering) may be used to group the multi-dimensional embedding vectors into smaller subsets. This may be used, for example, if the dataset of previously performed medical procedures is large.

It is noted that the matrix M is likely to be sparse e.g. most of the elements may be unknown or blank because not all of the previous medical professionals will have worked with (and thus provided a feedback rating for) all of the candidate medical professionals. Blank (e.g. unknown) entries of the matrix M may be found, for example, using matrix factorization methods known in the art, such as SVD, unconstrained or non-negative matrix factorization, or, using the machine learning approach described above and illustrated in FIG. 2.

It will be appreciated herein that the model may be updated, by acquiring further feedback on medical procedures in which the candidate medical professionals assist in. For example, the first medical professional may initiate a video call with the second medical professional to obtain assistance or insights into the medical procedure being performed by the first medical professional. Following the video call, the first medical professional may provide feedback indicating the relevance of the second medical professional to the medical procedure performed by the first medical professional. This feedback may be used to update the matrix or the model.

For example, in some embodiments, such as that illustrated in FIG. 2, the feedback may be used as ground truth data with which to provide further training to a neural network. In other embodiments such as that illustrated in FIG. 3, the feedback may be used as an additional data point in a matrix. This may improve clustering or other CF based methods described above.

In this manner, a second medical professional may be determined from a plurality of candidate medical professionals, as an expert with whom a first medical professional may want to contact for advice on a medical procedure being performed by the first medical professional.

In some embodiments, the system 100 may be further configured to initiate a call, such an audio-visual call between the first medical professional and the second medical professional. This may be performed, for example, with a communications interface 108.

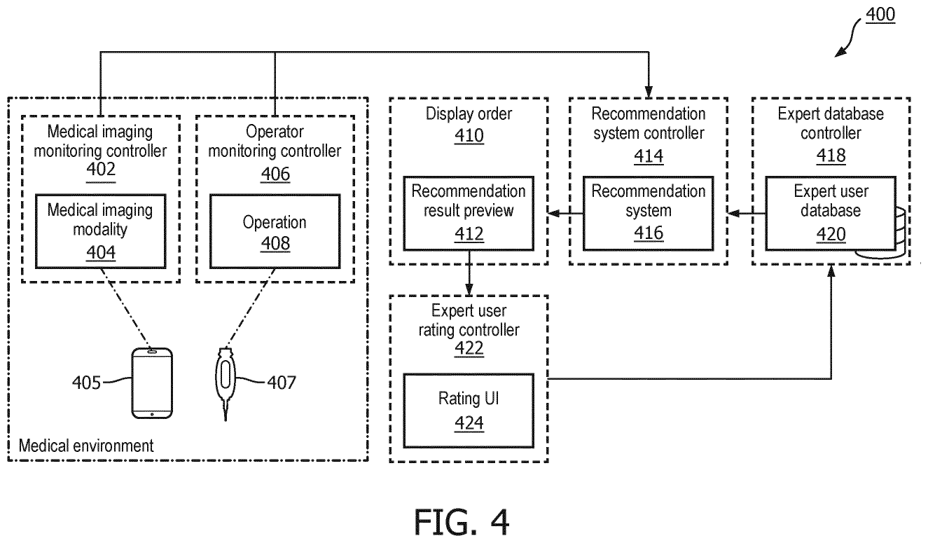

Turning to FIG. 4, which illustrates a system 400 according to some embodiments herein. In this embodiment, the system comprises a real-time recommendation system for a remote tele-collaboration module of a medical imaging modality, such as an ultrasound imager. The system 400 may be used by a first medical professional performing a medical procedure to obtain a recommendation of a second medical professional with experience relevant to the medical procedure being performed by the first medical professional, with whom the first medical professional may want to initiate a teleconferencing call, for example to obtain assistance from the second medical professional.

The system 400 comprises an medical imaging modality 404, such as, for example, a portable ultrasound device. An example of an ultrasound device is the “Lumify” portable ultrasound device, which operates in a controlled medical environment. Alternatively, medical imaging modality 404 may comprise, for example any one of:

a. a CT, MRI, or PET-CT scanner, a fixed or mobile X-ray system,

b. a 2D and/or 3D ultrasound device,

c. an interventional ultrasound catheter (intravascular, intracardiac ultrasound).

The medical imaging modality may further comprise a video or audio real-time tele-collaboration system integrated into the medical imaging modality. An example of such a tele-collaboration system is the collaboration feature (“Reacts”) on the aforementioned Lumify device. The medical imaging modality may interface with, a mobile phone or other device 405 capable of making an audio or visual call, such as a tablet or desktop computer.

The system 400 is configured to determine a first multi-dimensional embedding vector, as described above, in two stages. Firstly, the medical imaging monitoring (MIM) controller 402 is configured to determine an image-specific multi-dimensional embedding vector, from images taken by the medical imaging modality and/or a camera or other image recording equipment on the device 405. E.g. the images may comprise images taken by the camera of the first medical professional performing the medical procedure. Secondly, the Operator monitoring (OM) controller 406 is configured to extract one or more operational multi-dimensional embedding vectors relating to the operation of the imaging modality, such as for example, user-device characteristics. As is described in more detail below, the recommendation system (RS) 416 is configured to combine the image-specific multi-dimensional embedding vector and the operational multi-dimensional embedding vector(s) to determine the first multi-dimensional embedding vector.

The Expert Database (ED) controller 418 is configured to store information about previous medical procedures whereby a call was set up, (e.g. using the system 400) between a previous medical professional performing a previous medical procedure and a third medical professional who assisted with the previous medical procedure. For each such call made using the system 400, the ED controller 418 is configured to determine a third multi-dimensional vector describing the third medical professional who assisted with the previous medical procedure. The third medical professional is then added to a list of candidate medical professionals with whom a call may be set up with. The third multi-dimensional embedding vector may encode, for example, the years of experience, primary area of expertise (abdominal, cardiac, gastric, focused assessment with sonography in trauma—FAST exam), and/or length of previous remote sessions aided by the third medical professional.

The Expert user rating (EUR) controller 422 comprises a rating user interface (UI) 424 which is displayed to the medical professional performing the medical procedure and is configured to collect a feedback ratings from the medical professional on the assistance (e.g. relevance) of the assisting (e.g. second) medical professional, as soon as a remote session is terminated. As noted above, the feedback rating may describe the degree of satisfaction with the interaction with the remote, assisting (e.g. second) medical professional. The EUR controller 422 send collected ratings to a recommendation system (RS) controller 414.

The RS controller 414 may be configured to receive in real-time the multi-dimensional embedding vectors from both MIM and OM controllers and concatenate them into one dense multi-dimensional embedding vector, referred to herein as the first multi-dimensional embedding vector. The RS controller is further configured to pass the first multi-dimensional embedding vector and the plurality of candidate multi-dimensional embedding vectors to the recommendation system 416. The recommendation system 416 is trained to determine a second medical professional from the plurality of candidate medical professionals, based on the first multi-dimensional embedding vector and the plurality of candidate multi-dimensional embedding vectors, with whom the first medical professional may want to initiate a call with. In other words, the recommendation system 416 may recommend one or more expert users, whose advices might be the most valuable for the operator of the imaging modality based on the received multi-dimensional embedding vectors. The model may be used to determine a second medical professional, according to any of the methods described above with respect to FIGS. 1-3.

The recommender system may further check the online availability of the determined second medical professional, and display the second medical professional using the display controller 410. The recommendation system may recommend a plurality of candidate medical professionals, in the form of an ordered list, ordered according to predicted relevance.

The Display controller 410 may be configured to: receive details of the second medical professional (or an ordered subset of the candidate medical professionals, ordered by relevance) from the RS controller 414 and present the second medical professional to the user (i.e. the first medical professional).

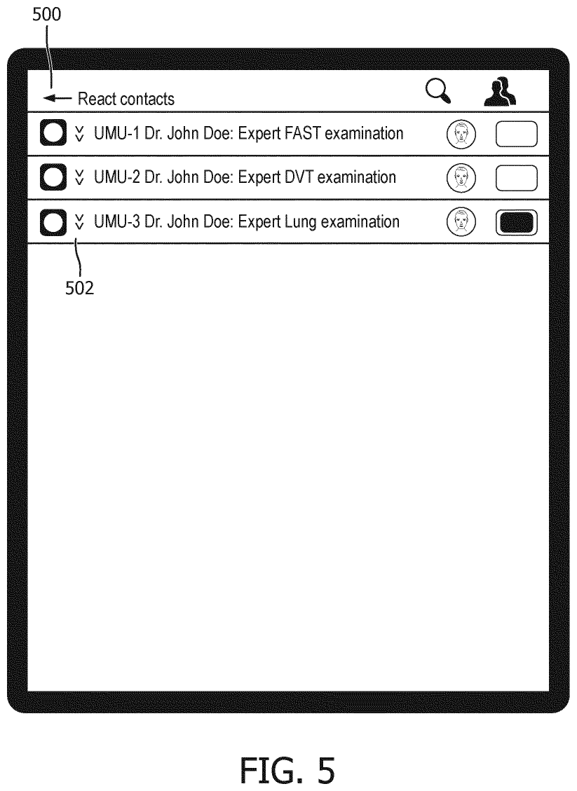

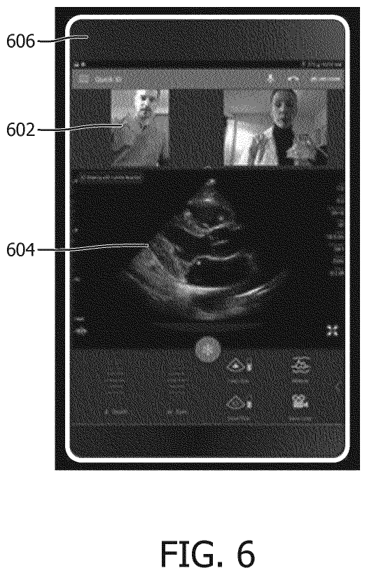

FIG. 5 illustrates an example graphical user interface (GUI) 500 showing an ordered list of candidate medical professionals 502, ordered according to the recommendation system of FIG. 4. The GUI 500 may be displayed, for example by the display controller 410. FIG. 6 illustrates a further GUI, showing an example GUI of a medical teleconference call according to embodiments herein. In this embodiment, a first medical professional 602 performing a medical procedure comprising an ultrasound scan 604 is aided by a second medical professional 606 (who was determined or matched to the first medical professional, according to the methods described herein). The first medical professional performs the ultrasound scan using an ultrasound scanner and images from the scan, along with a video link are sent to the second medical professional who can view them on a remote device, e.g. such as a laptop.

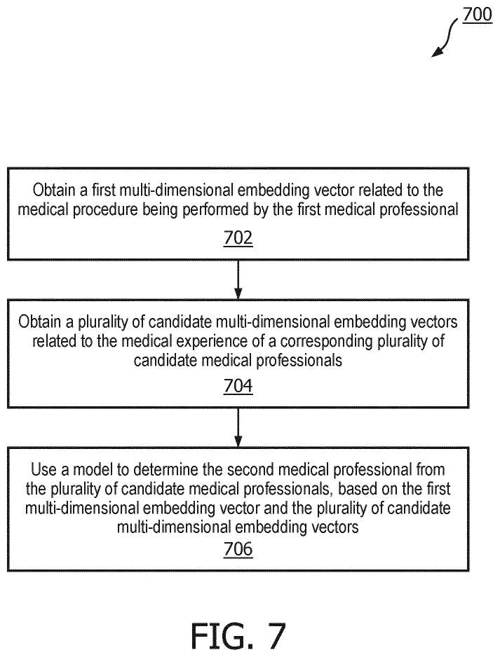

FIG. 7 illustrates a computer implemented method 700 of determining a second medical professional having experience relevant to a medical procedure being performed by a first medical professional, according to some embodiments herein. Briefly, the method 700 comprises: obtaining 702 a first multi-dimensional embedding vector related to the medical procedure being performed by the first medical professional; obtaining 704 a plurality of candidate multi-dimensional embedding vectors related to medical experience of a corresponding plurality of candidate medical professionals; and using a model to determine the second medical professional from the plurality of candidate medical professionals, based on the first multi-dimensional embedding vector and the plurality of candidate multi-dimensional embedding vectors.

The method 700 may be performed, for example, by the system 200 or the system 400 as described above. The method 700 may be used, for example, as part of a medical telecommunications system, to match a second medical professional with the first medical professional, and to recommend the second medical professional to the first medical professional as someone with whom the first medical professional may want to seek assistance from (e.g. initiate a video call with) when performing the medical procedure.

The method 700 may also be used, for example, as part of a review based system. For example, the second medical professional may comprise a reviewer having experience relevant to a medical procedure being performed by a first medical professional who can review the performance of the first medical professional, e.g. peri-interventional review by a colleague physically present at the hospital facility (who was notified by the system), or post-procedural review by an expert at the end of the day.

Obtaining a first multi-dimensional embedding vector related to the medical procedure being performed by the first medical professional was described in detail above with respect to the systems 200 and 400 the detail therein will be understood to apply equally to the method 700.

Obtaining a plurality of candidate multi-dimensional embedding vectors related to medical experience of a corresponding plurality of candidate medical professionals was described in detail above with respect to the systems 200 and 400 and the detail therein will be understood to apply equally to the method 700.

Using a model to determine the second medical professional from the plurality of candidate medical professionals, based on the first multi-dimensional embedding vector and the plurality of candidate multi-dimensional embedding vectors was described in detail above with respect to the system 200 and the detail therein will be understood to apply equally to the method 700.

In some embodiments, the method may further comprise initiating a video-conferencing call between the first medical professional and the second medical professional.

FIG. 8 shows a system 800 that may comprise an extension to the system 400 (as described above) according to some embodiments herein. The proposed recommendation system 400 may result in the same user(s) being repeatedly determined (or selected) to assist with medical procedures. For example, users who are regularly available and/or highly specialized in a common medical procedure type. The system of FIG. 8 proposes a gratification system for rewarding expert users with the highest feedback relevance ratings and/or work-loads. The system 800 for this embodiment comprises an Expert database controller (ED) 802. The ED 802 may comprise the ED 418 as described above with respect to FIG. 4. The system 800 further comprises a Broadcasting controller 804 configured to broadcast the saved ultrasound loops resulting from a videoconferencing call between the first and second medical professionals to the registered user list.

There is further an Expert user rating (EUR) controller 806 as described above with respect to the EUR 422 of FIG. 4. The EUR 806 is further configured to assist in determining a feedback rating for the second medical professional (e.g. the assisting medical professional), based on a comparison between a diagnosis determined by a remote diagnosis controller 808 and a diagnosis determined as part of the video call between the first and second medical professionals. The remote diagnosis controller 808 is also configured to transcript the diagnosis outcome for the broadcasted loop, and implement a payment mechanism system for the assisted expert (if the clinical workflow permits).

In another embodiment, there is provided a computer program product comprising a computer readable medium, the computer readable medium having computer readable code embodied therein, the computer readable code being configured such that, on execution by a suitable computer or processor, the computer or processor is caused to perform the method or methods described herein.

Thus, it will be appreciated that the disclosure also applies to computer programs, particularly computer programs on or in a carrier, adapted to put embodiments into practice. The program may be in the form of a source code, an object code, a code intermediate source and an object code such as in a partially compiled form, or in any other form suitable for use in the implementation of the method according to the embodiments described herein.

It will also be appreciated that such a program may have many different architectural designs. For example, a program code implementing the functionality of the method or system may be sub-divided into one or more sub-routines. Many different ways of distributing the functionality among these sub-routines will be apparent to the skilled person. The sub-routines may be stored together in one executable file to form a self-contained program. Such an executable file may comprise computer-executable instructions, for example, processor instructions and/or interpreter instructions (e.g. Java interpreter instructions). Alternatively, one or more or all of the sub-routines may be stored in at least one external library file and linked with a main program either statically or dynamically, e.g. at run-time. The main program contains at least one call to at least one of the sub-routines. The sub-routines may also comprise function calls to each other.

The carrier of a computer program may be any entity or device capable of carrying the program. For example, the carrier may include a data storage, such as a ROM, for example, a CD ROM or a semiconductor ROM, or a magnetic recording medium, for example, a hard disk. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

Variations to the disclosed embodiments can be understood and effected by those skilled in the art, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A method of determining a second medical professional having experience relevant to a medical procedure being performed by a first medical professional the method comprising:
obtaining a first multi-dimensional embedding vector related to the medical procedure being performed by the first medical professional;
obtaining a plurality of candidate multi-dimensional embedding vectors related to medical experience of a corresponding plurality of candidate medical professionals; and
using a model to determine the second medical professional from the plurality of candidate medical professionals, based on the first multi-dimensional embedding vector and the plurality of candidate multi-dimensional embedding vectors.

2. The method as in claim 1 wherein the method is performed by a medical teleconferencing system and wherein the method further comprises:
recommending the second medical professional to the first medical professional as a person with whom to initiate a video call in order to obtain remote assistance with the medical procedure.

3. The method as in claim 1 wherein the model comprises a model trained using a machine learning process to take as input the first multi-dimensional embedding vector and a candidate multi-dimensional embedding vector from the plurality of candidate multi-dimensional embedding vectors, and output a relevance value for the respective candidate medical professional, wherein the relevance value comprises a prediction of the relevance of the respective candidate medical professional to the medical procedure being performed by the first medical professional.

4. The method as in claim 3 wherein the model has been trained using training data comprising: i) a multi-dimensional embedding vector related to an example medical procedure ii) a multi-dimensional embedding vector related to medical experience of an example medical professional; and iii) a ground truth relevance value comprising a measure of relevance of the example medical professional to the example medical procedure.

5. The method as in claim 4 wherein the ground truth relevance value comprises user feedback from a third medical professional with respect to the relevance of assistance provided by the example medical professional to the example medical procedure.

6. The method as in claim 3 wherein the method further comprises:
displaying a list comprising a subset of the plurality of candidate medical professionals to the first medical professional, the list being ordered according to relevance values of the subset of the plurality of candidate medical professionals as determined by the model.

7. The method as in claim 1 wherein the model comprises a neural network.

8. The method as in claim 1, wherein the step of using a model to determine the second medical professional from the plurality of candidate medical professionals is further based on:
information relating to a plurality of previously performed medical procedures, the information comprising, for each of the previously performed medical procedures:
a multi-dimensional embedding vector, relating to a previous medical professional who performed the previous medical procedure;
an indication of a third medical professional from the plurality of candidate medical professionals who assisted with the previous medical procedure; and
a feedback rating provided by the previous medical professional that rates the assistance provided by the third medical professional to the previous medical procedure.

9. The method as in claim 8 wherein the step of using a model to determine the second medical professional from the plurality of candidate medical professionals comprises using collaborative filtering to determine the second medical professional based on the first multi-dimensional embedding vector and the information relating to the plurality of previously performed medical procedures.

10. The method as in claim 1 further comprising:
receiving feedback from the first medical professional following a video call between the first and second medical professionals, the feedback indicating a relevance of the second medical professional to the medical procedure performed by the first medical professional; and
using the received feedback to update the model.

11. The method as in claim 1 wherein the medical procedure comprises an ultrasound examination.

12. The method as in claim 1 wherein the first multi-dimensional embedding vector comprises information relating to:
a signature of an image of the medical procedure being performed by the first medical professional, the signature being obtained from a feed-forward pass over a convolutional neural network, CNN;
an image quality of a video stream of the procedure being performed by the first medical professional;
an anatomical feature visible in a video stream of the procedure being performed by the first medical professional;
a diagnostic evaluation of the medical procedure as deduced from a video stream of the procedure being performed by the first medical professional.
an indication of the experience of the first medical professional; and/or
an indication of the environment in which procedure is being performed by the first medical professional.

13. A system for determining a second medical professional having experience relevant to a medical procedure being performed by a first medical professional, the system comprising:
a memory comprising instruction data representing a set of instructions; and
a processor configured to communicate with the memory and to execute the set of instructions, wherein the set of instructions, when executed by the processor, cause the processor to:
obtain a first multi-dimensional embedding vector related to the medical procedure being performed by the first medical professional;
obtain a plurality of candidate multi-dimensional embedding vectors related to medical experience of a corresponding plurality of candidate medical professionals; and
use a model to determine the second medical professional from the plurality of candidate medical professionals, based on the first multi-dimensional embedding vector and the plurality of candidate multi-dimensional embedding vectors.

14. The system as in claim 13 wherein system comprises a medical teleconferencing system and wherein the processor is further caused to:
recommend the second medical professional to the first medical professional as a person with whom to initiate a video call in order to obtain remote assistance with the medical procedure.

15. A non-transitory computer program product comprising computer readable medium, the computer readable medium having computer readable code embodied therein, the computer readable code being configured such that, on execution by a suitable computer or processor, the computer or processor is caused to perform the method as claimed in claim 1.
